(19) United States
(12) Patent Application Publication
(10) Pub. No.: US 2011/0275877 A1
(43) Pub. Date: Nov. 10, 2011

(54) Separation of Normal Paraffins from Isoparaffins Using Rapid Cycle Pressure Swing Adsorption

(75) Inventors: Bal K. Kaul, Fairfax, VA (US); John H. Thurtell, Centreville, VA (US); Jose G. Santiesteban, Hellertown, PA (US); Jasmina Poturovic, Vienna, VA (US)

(73) Assignee: EXXONMOBIL RESEARCH AND ENGINEERING COMPANY, Annandale, NJ (US)

(21) Appl. No.: 13/080,770

(22) Filed: Apr. 6, 2011

Related U.S. Application Data
(60) Provisional application No. 61/395,161, filed on May 7, 2010.

(51) Int. Cl. C07C 7/13; C07C 7/12

(52) U.S. Cl. 585/820

(57) ABSTRACT

The separation of normal paraffins from isoparaffins using rapid cycle pressure swing adsorption. The present invention also relates to an isomerization process wherein normal paraffins are converted to isoparaffins resulting in an effluent stream containing a mixture of normal paraffins and isoparaffins, which effluent stream is sent to a rapid cycle pressure swing adsorption unit to perform the separation of normal paraffins from isoparaffins.

This application claims the benefit of U.S. Provisional Application No. 61/395,161 filed May 7, 2010.

### FIELD OF THE INVENTION

The present invention relates to the separation of normal from isoparaffins using rapid cycle pressure swing adsorption. The present invention also relates to an isomerization process wherein normal paraffins are converted to isoparaffins resulting in an effluent stream containing a mixture of normal paraffins and isoparaffins, which effluent stream is sent to a rapid cycle pressure swing adsorption unit to perform the separation of normal paraffins from isoparaffins.

### BACKGROUND OF THE INVENTION

Conventional gasoline blending pools typically include C₄ and heavier hydrocarbons having boiling points of less than about 205° C. (395° F.) at atmospheric pressure. This range of hydrocarbons includes C₄-C₆ paraffins, especially C₅ and C₆ normal paraffins that have relatively low octane numbers. Since the phase-out of lead additive octane improvers many years ago, higher octane gasolines have been produced using isomerization to rearrange the structure of paraffinic hydrocarbons to branched paraffins, or reforming to convert C₆ and heavier hydrocarbons to aromatics. Normal C₅ hydrocarbons are not readily converted to aromatics, therefore, conventional practice has been to isomerize these lighter hydrocarbons into the corresponding branched isoparaffins. Although C₆ and heavier hydrocarbons can be upgraded to aromatics through hydrocyclization, the conversion of C₆ hydrocarbons to aromatics creates higher density species and increases gas yields, thus resulting in a reduction of liquid volume yields. Moreover, health concerns related to benzene are likely to generate overall restrictions of benzene and possibly for other aromatics as well, which some view as precursors for benzene tail pipe emissions. Therefore, it is desirable to conduct the C₆ normal paraffins to an isomerization unit to produce C₆ isoparaffins.

The effluent from an isomerization reaction zone will typically contain a mixture of more highly branched paraffins, less highly branched paraffins, and normal paraffins. In order to increase the octane of the product stream from an isomerization zone, normal paraffins, and sometimes at least a portion of the less highly branched isoparaffins, are separated from the more highly branched isoparaffins and recycled to the isomerization zone in order to increase the ratio of more highly branched paraffins to less branched paraffins entering the isomerization zone. A variety of conventional methods are known for treating the effluent from an isomerization zone to separate normal paraffins and less highly branched isoparaffins, such as monomethyl-branched isoparaffins, for recycling. However, there exists a need in the art for more cost effective technologies for separating normal paraffins from isoparaffins.

### SUMMARY OF THE INVENTION

In a preferred embodiment, the present invention relates to a method for the separation of normal paraffins from isoparaffins from a hydrocarbon-containing feedstream that contains both normal paraffins and isoparaffins, which method comprising passing said feedstream to a rapid cycle pressure swing adsorption unit having a cycle time less than about 1 minute and containing an adsorbent material capable of adsorbing at least an effective amount of normal paraffins from said stream and separately collecting a normal paraffin-rich effluent stream and an isoparaffin-rich effluent stream rich.

The term “normal paraffin-rich effluent stream” as used herein, means a rapid cycle pressure swing adsorption unit hydrocarbon product stream that has a higher wt % of normal paraffins than said hydrocarbon feedstream to the rapid cycle pressure swing adsorption unit, unless such term is further limited. Similarly, the term “isoparaffin-rich effluent stream” as used herein, means a rapid cycle pressure swing adsorption unit hydrocarbon product stream that has a higher wt % of isoparaffins than said hydrocarbon feedstream to the rapid cycle pressure swing adsorption unit, unless such term is further limited.

In another preferred embodiment of the present invention, the normal paraffin-rich effluent stream contains at least 25 wt % more normal paraffins than said hydrocarbon feedstream. In another preferred embodiment of the present invention, the isoparaffin-rich effluent stream contains at least 25 wt % more isoparaffins than said hydrocarbon feedstream.

In a preferred embodiment the adsorbent is present as a structured material selected from monoliths and sheets.

In another preferred embodiment, the adsorbent material is selected from the group consisting of crystalline molecular sieves, activated carbons, activated clays, silica gels, activated aluminas and zeolites.

In preferred embodiments, the adsorbent material is a zeolite with a zeolitic framework selected from CHA, HEU, ERI, FAU, FER, MOR, LTA, and KFI. More preferably the zeolite is of a framework is selected from CHA, FAU, FER, and LTA. Most preferably, the zeolitic framework is CHA. In preferred embodiments, the adsorbent material is a zeolite selected from Chabazite, Linde D, Clinoptilolite, Erionite, Faujasite, Linde X, Linde Y, Ferrierite, ZSM-35, Mordenite, Linde A, and Linde P. More preferably, the zeolite is selected from Chabazite and Linde D.

In most preferred embodiments, the adsorbent material is a zeolite having a silica to alumina ratio (Si:Al) greater than about 50, preferably greater than about 100, and more preferably greater than about 1000.

In still another preferred embodiment the stream containing the normal paraffins and isoparaffins is an effluent stream from an isomerization process unit.

### BRIEF DESCRIPTION OF THE FIGURES

FIG. 1 hereof is a process flow schematic of an alkylation process unit producing 13,000 bpd alkylation product from feed received from a Butamer™ process unit having a capacity of 3,900 bpd of butane feed.

FIG. 2 hereof is a process flow schematic similar to that of FIG. 1 hereof but with a separation of normal paraffins from isoparaffins between the Butamer™ and alkylation process units, which separation is performed by use of a rapid cycle pressure swing adsorption unit.

FIG. 3 hereof is a process flow schematic of an alkylation process unit receiving saturated gas purge (SGP) as a feed.

FIG. 4 hereof is a process flow schematic similar to that of FIG. 3 hereof, but with the separation of normal and isoparaffins of the saturated gas purge stream upstream of the alkylation process unit, which separation is performed by use of a rapid cycle pressure swing adsorption unit.

### DETAILED DESCRIPTION OF THE INVENTION

The present invention relates to the use of rapid cycle pressure swing adsorption (RCPSA) for separating normal paraffins from isoparaffins from gaseous streams containing both normal and isoparaffins. The duration of the cycle of a rapid cycle pressure swing adsorption unit, compared to a conventional PSA unit, is significantly less. Conventional pressure swing adsorption is well known in the art. In it, a gaseous mixture is passed, under pressure, through a fixed-bed of a solid sorbent material that is selective for adsorbing one or more components of the gaseous mixture. For convenience, the component or components that are to be removed from a gaseous mixture will be referred to herein in the singular and sometimes referred to as a contaminant. The gaseous mixture is passed through a sorption bed and emerges from the bed depleted in the contaminant that remains sorbed in the bed. After a predetermined period of time, or alternatively, when break-through of the contaminant is observed, the flow of gas is switched to another sorbent bed in a separate vessel for the separation to continue. At the same time, the sorbed contaminant is removed from the original bed by a reduction in pressure, usually accompanied by a reverse flow of gas to desorb the contaminant. As the pressure in the vessel is reduced, the contaminant previously adsorbed on the bed of sorbent is progressively desorbed into the tail gas system that typically comprises a large tail gas drum, together with a control system designed to minimize pressure fluctuations to downstream systems. Because of rapid cycling of an RCPSA unit, the use of a surge drum can be eliminated since rapid cycling provides a continuous flow of a desorbed n-paraffin stream. The contaminant can be collected from the tail gas system in any suitable manner and further processed or disposed of as appropriate. When desorption is complete, the sorbent bed can be purged with an inert gas, e.g., nitrogen or a purified stream of the process gas. Purging can be facilitated by the use of a heated purge gas stream. Thus, a pressure swing cycle will typically include a feed step, a depressurization step, a purge, and then finally repressurizing to prepare for the feed step of another cycle.

After breakthrough in the second bed, and after the first bed has been regenerated so that it is again prepared for adsorption service, the flow of gaseous mixture is switched from the second bed to the first bed, and the second bed is regenerated. The total cycle time is the length of time from when the gaseous mixture is first conducted to the first bed in a first cycle to the time when the gaseous mixture is first conducted to the first bed in the immediately succeeding cycle, i.e., after a single regeneration of the first bed. The use of third, fourth, fifth, etc. vessels in addition to the second vessel, as might be needed when adsorption time is short but desorption time is long, can serve to increase cycle time.

Conventional PSA is not suitable for use in the present invention for a variety of reasons. Not only is it too costly, but a conventional PSA unit will have cycle times in excess of one minute, typically in excess of 2 to 4 minutes. A rapid cycle pressure swing adsorption unit used in the practice of the present invention will have cycle times of less than about one minute, typically less than about 30 seconds, preferably less than about 10 seconds and more preferably less than about 1 second.

Further, the rapid cycle pressure swing adsorption units used in the practice of the present invention can have substantially different sorbents than do conventional PSA units, which sorbents will typically be comprised of structured materials, such as monoliths. Use of structured adsorbents, or monoliths, also have an advantage of maintaining rigidity (substantially no attrition) which is often a problem with conventional PSA sorbents. In addition, because of fast cycle times, a much smaller volume of sorbent is used when compared to conventional PSA.

One preferred type of adsorbent used in the practice of the present invention is in the form of adsorbent sheets comprised adsorbent material coupled to a structured reinforcement material. A suitable binder can be used to attach the adsorbent material to the reinforcement material. Non-limiting examples of reinforcement material include monoliths, a mineral fiber matrix, (such as a glass fiber matrix), a metal wire matrix (such as a wire mesh screen), or a metal foil (such as aluminum foil), which can be anodized. Examples of glass fiber matrices include woven and non-woven glass fiber scrims. The adsorbent sheets can be made by coating with a slurry of suitable adsorbent component, such as zeolite crystals with binder constituents onto the reinforcement material, such as non-woven fiber glass scrims, woven metal fabrics, and expanded aluminum foils.

An absorber in a rapid cycle pressure swing adsorption unit typically comprises an adsorbent solid phase formed from one or more adsorbent materials and a permeable gas phase through which the gases to be separated flow from the inlet to the outlet of the adsorber. Components to be removed are fixed on the solid phase. This gas phase is typically called “circulating gas phase” or more simply “gas phase”. The solid phase includes a network of pores, the mean size of which is generally between about 0.020 μm and about 20 μm, this being called a “macropore network”. There may be a network of even smaller pores in the micropore range. A micropore network will be encountered, for example, in microporous carbon adsorbents or zeolites. As previously mentioned, the solid phase can be deposited on a non-adsorbent support, the function of which is to provide mechanical strength or support. The support can also play a thermal conduction role or to store heat. The phenomenon of adsorption comprises two main steps, namely passage of the adsorbate from the circulating gas phase onto the surface of the solid phase, followed by passage of the adsorbate from the surface to the volume of the solid phase into the adsorption sites.

Rapid cycle pressure swing absorption may utilize a rotary valving system to conduct the gas flow through a rotary sorber module that contains a number of separate compartments, each of which is successively cycled through the sorption and desorption steps as the rotary module completes the cycle of operations. By use of a suitable arrangement of the valving, a number of individual compartments can be passing through the characteristic steps of the complete cycle at any given time. Flow and pressure variations required for the sorption/desorption cycle can be changed in increments in order to smooth-out the pressure and flow rate pulsations encountered by the compression and valving machinery. In this form, the rapid cycle pressure swing adsorption module includes valving elements angularly spaced around the circular path taken by the rotating sorption module. Each compartment is successively passed to a gas flow path in the appropriate direction and pressure to achieve one of the incremental pressure/flow direction steps in the complete rapid cycle pressure swing adsorption cycle used. The key advantage of the rapid cycle pressure swing adsorption technology is a more efficient use of sorbent material. In fact, the quantity of sorbent material required with rapid cycle pressure swing adsorption technology is typically only a fraction of that required for conventional PSA technology (for the same contaminant separation service). As a result, the footprint and investment required for rapid cycle pressure swing adsorption is lower than that for conventional PSA.

U.S. Pat. Nos. 6,406,523; 6,451,095; 6,488,747; 6,533,846; 6,565,635; and 7,591,879 all of which are incorporated herein by reference, teach various aspects of rapid cycle pressure swing adsorption technology.

As previously mentioned, the rapid cycle pressure swing adsorption process of the present invention is preferably associated with an isomerization process unit wherein a predominantly C₅ and C₉ normal paraffin stream is isomerized in the presence of an isomerization catalyst at isomerization conditions resulting in the conversion of a substantial amount of the normal paraffins to isoparaffins. The isoparaffins, in comparison to the normal paraffins, are substantially higher in octane value for blending in a refinery gasoline pool. Isomerization is also used to convert normal C₄'s to isobutanes for use in an alklylation plant for the production of octane improver additives.

Contacting within the isomerization zones can be effected using any suitable catalyst bed system. Non-limiting examples of types of catalyst beds suitable for use herein include: fixed-bed systems, moving-bed systems, fluidized-bed systems. A fixed-bed system is preferred. The reactants can be contacted with the bed of catalyst particles in either an upward, downward, or radial-flow fashion. Further, the reactants can be in the liquid phase, a mixed liquid-vapor phase, or a vapor phase when contacted with the catalyst particles. A primarily liquid-phase operation is preferred. The isomerization zone can be in a single reactor or in two or more separate reactors with suitable means to ensure that the desired isomerization temperature is maintained at the entrance to each zone. Two or more reactors in sequence are preferred to enable improved isomerization through control of individual reactor temperatures and for partial catalyst replacement without a process shutdown.

Isomerization conditions in the isomerization zone include reactor temperatures ranging from about 25° C. to 300° C. Lower reaction temperatures are generally preferred in order to favor equilibrium mixtures having the highest concentration of high-octane highly branched isoalkanes and to minimize cracking of the feed to lighter hydrocarbons. Temperatures in the range of about 100° C. to about 250° C. are preferred. Reactor operating pressures generally range from about 100 kPa to 10 Mpa absolute, preferably between about 0.3 Mpa and 4 Mpa. Liquid hourly space velocities range from about 0.2 to about 25 v/v/hr, preferably from about 0.5 to 10 v/v/hr.

Hydrogen is admixed with, or remains with, the paraffinic feedstock to the isomerization zone to provide a mole ratio of hydrogen to hydrocarbon feed from about 0.01 to 20, preferably from about 0.05 to 5. The hydrogen can be supplied totally from outside the process or supplemented by hydrogen recycled to the feed after separation from the reactor effluent. Light hydrocarbons and small amounts of inert material such as nitrogen and argon can be present in the hydrogen stream. Water, if present, is preferably removed from hydrogen stream supplied from outside the process. Although any water can be removed by any suitable method, it is preferred that it be removed by an adsorption process, which is well known in the art. In a preferred embodiment, the hydrogen to hydrocarbon mole ratio in the reactor effluent is equal to or less than about 0.05, generally obviating the need to recycle hydrogen from the reactor effluent to the feed. Upon contact with the catalyst, at least a portion of the paraffinic feedstock is converted to desired, higher octane, isoparaffin products.

The present invention can be carried out using any suitable sorbent material in the rapid cycle pressure swing adsorption unit that has capacity for the selective sorption of either isoparaffin or the normal paraffin components. It is preferred that there be no chemical reaction with the sorbent since this will increase the difficulty of achieving desorption of any contaminant that has become chemically bound to the sorbent. That said, chemisorption is not to be excluded in the practice of the present invention if the adsorbed contaminant can be effectively desorbed, e.g., by the use of higher temperatures coupled with the reduction in pressure.

Suitable sorbents known in the art are those that are commercially available and include crystalline molecular sieves, activated carbons, activated clays, silica gels, activated aluminas and the like. The molecular sieves include, for example, the various forms of silicoaluminophosohates and aluminophosphates disclosed in U.S. Pat. Nos. 4,440,871; 4,310,440 and 4,567,027, all of which are incorporated herein by reference. Zeolitic molecular sieves are also suitable for use herein.

In preferred embodiments, zeolites which can be used in the practice of the present invention include a selection from the following zeolitic frameworks: CHA, HEU, ERI, FAU, FER, MOR, LTA, and KFI. More preferably the zeolite is of a framework is selected from CHA, FAU, FER, and LTA. Most preferably, the zeolitic framework is CHA. In preferred embodiments, zeolites which can be used in the practice of the present invention include a selection from the following zeolites: Chabazite, Linde D, Clinoptilolite, Erionite, Faujasite, Linde X, Linde Y, Ferrierite, ZSM-35, Mordenite, Linde A, and Linde P. More preferably the zeolite is selected from Chabazite, Linde D, Faujasite, Linde X, Linde Y, Ferrierite, ZSM-35, and Linde A. Most preferably, the zeolite is selected from Chabazite and Linde D.

Preferably, zeolites are utilized in the present invention and the zeolites have a high silica content, i.e., a silica to alumina ratio (Si:Al) greater than about 50, preferably greater than about 100, and more preferably greater than about 1000. One such high silica zeolite is silicalite which includes both the silicapolymorph disclosed in U.S. Pat. No. 4,061,724 as well as the F-silicate disclosed in U.S. Pat. No. 4,073,865, both of which are incorporated herein by reference. Detailed descriptions of some of the above-identified zeolites can be found in D. W. Breck, Zeolite Molecular Sieves, John Wiley and Sons, New York, 1974, which is also incorporated herein by reference. Other preferred sorbents include type 5A molecular sieves, more preferably in the form of ⅛ pellets. The selection of other adsorbents for normal hydrocarbon separation can be made by one skilled in the art by routine experimentation.

It is often desirable when using crystalline molecular sieves that the molecular sieve be agglomerated with a binder in order to ensure that the sorbent will have suitable physical properties. There are a variety of synthetic and naturally occurring binder materials available for use in the present invention. Non-limiting examples of such binder materials include metal oxides, clays, silicas, aluminas, silica-aluminas, silica-zirconias, silica thorias, silica-berylias, silica-titanias, silica-aluminas-thorias, silica-alumina-zirconias, mixtures of these and the like. Clay-type binders are preferred. Non-limiting examples of clay type binders that can be used to agglomerate the molecular sieve without substantially altering the sorption properties of the sorbent include attapulgite, kaolin, volclay, sepiolite, polygorskite, kaolinite, bentonite, montmorillonite, illite and chlorite. The choice of a suitable binder and methods employed to agglomerate the sorbent are generally known to those skilled in the art.

Temperatures used in the sorption process of the present invention are not critical, although in general, the process is substantially isothermal. Typical temperatures will range from about 10° C. to about 400° C., preferably from about 90° C. to about 320° C., and more preferably from about 175° C. to about 230° C. The temperature for carrying out the separation is also dictated by the feed. The temperature of choice will be above the dew point of the feed mixture so that separation can be carried out in the vapor phase. It is preferred that all steps in a cycle be performed at substantially the same temperature. It is to be understood, however, that even though the process is generally isothermal, there is to be expected a certain degree of temperature variation associated with the thermal effects of the heats of adsorption and desorption.

Similarly, pressure levels employed during the rapid cycle pressure swing adsorption process are not critical provided that the pressure differential between the adsorption and desorption steps is sufficient to cause a change in the adsorbate fraction loading on the adsorbent thereby providing a delta loading effective for separating the targeted contaminant. Typical pressure levels during the sorption step will range from about 50 to 2000 psia, preferably from about 80 to 500 psia, and more preferably from about 80 to about 300 psia. Typical pressure levels at the end of the final desorption step will range from about 0.5 to about 200 psia, preferably from about 0.5 to 50 psia, and more preferably from about 0.5 to about 10 psia. Pressures during any equalization or blowdown steps, purge step, first countercurrent desorption step and countercurrent purge step will be intermediate between the sorption the final desorption steps. Co-current venting can be used to reduce the adsorbent bed pressure to within a range of 30 to 15 psia.

In general, the total cycle time, that is, the time required to perform all at the individual steps in a rapid cycle pressure swing adsorption cycle ranges from milliseconds to about 1 minute, preferably from about 1 second to about 30 seconds, more preferably less than about 10 seconds, and most preferably less than about 1 second. At least two sorbent beds are required in order to perform each equalization step and typically at least three sorbent beds and one additional vessel is preferred in order to provide a constant source of product gas.

The following examples will serve to illustrate, but not limit, this invention.

### Example 1

Isotherm data of n-butane was obtained on a molecular sieve (5A) using a Micromeritics™ (Model ASAP2010) adsorption unit. The relative adsorption capacity (grams of butane/100 grams of adsorbent) was obtained by standard procedures. Standard commercially available equipment (gravimetric or volumetric) can be used for adsorption measurement. Commercially available highly exchanged 5A powered adsorbent was used to measure capacity (grams/100 grams of adsorbent) of n-butane. Increase in weight of sorbent was measured in standard gravimetric equipment (e.g., Micromeritics™) as a function of butane pressure and at various temperatures as reported in table below. The sorbent was heated to high temperature (greater than 350° C.) and purged with nitrogen gas to remove any moisture adsorbed onto the adsorbent before carrying out any measurements with butane. Table 1 below provides experimental data that shows the feasibility of carrying out separation of isoparaffin versus normal paraffins.

TABLE 1

| | | Capacity |
|---|---|---|
| Temperature | Pressure of butane | (grams/100 grams of |
| (° C.) | (torr) | adsorbent) |
| 148.2 | 800 | 8.9 |
| 148.2 | 100 | 2.8 |
| 248.0 | 800 | 5.6 |
| 248.0 | 100 | 2.1 |

The above data illustrates that at a constant temperature, reducing the partial pressure of butane from 800 to 100 torr provides a good working capacity for rapid cycle pressure swing adsorption. For example, at 148.2° C., a working capacity of 6 grams of butane is available and at 248° C. a working capacity of 3.5 grams/100 grams of adsorbent is available to carry out the necessary separation. Rapid cycle pressure swing adsorption technology relies on fast cycle and substantially the same small volume of sorbent is used repeatedly. Lower working capacity does not make it impractical to separate normal paraffins from isoparaffins as would be the case with conventional commercial technology, such as the Isosieve™ Process in which a molecular sieve made-up of a synthesized zeolite having 5 Å pores is used in which adsorption/desorption of n-paraffins onto the molecular sieve is carried out by repeating application and release of a pressure alternately. It should be noted that because of molecular diameter of isoparaffins being greater than 5 Å, the capacity of the sorbent for isoparaffin was essentially close to zero.

### Example 2

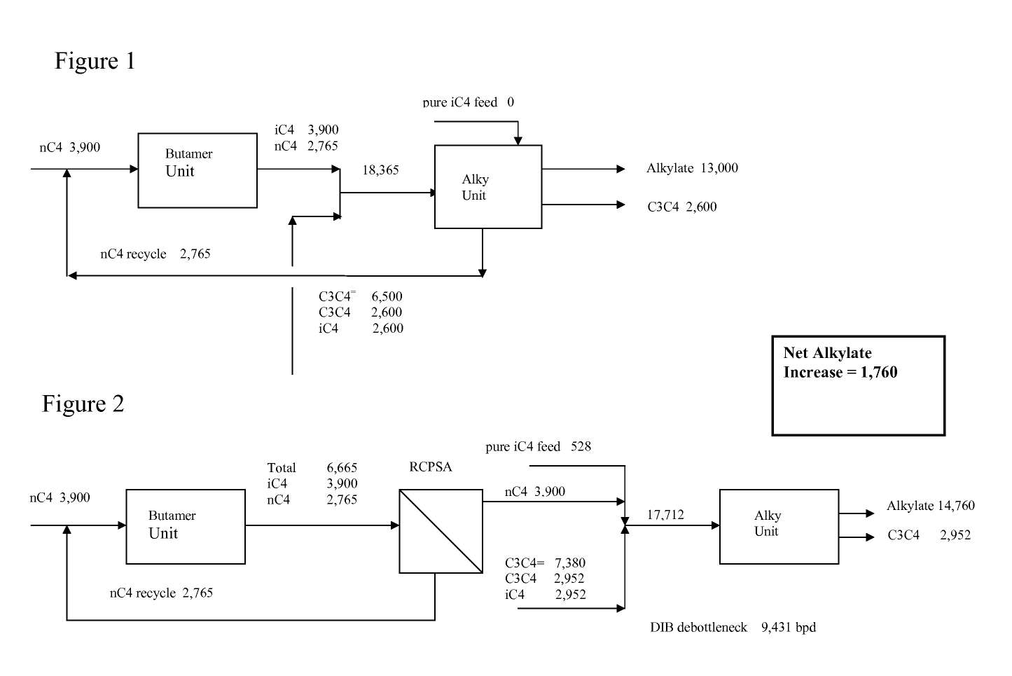

The data presented in Examples 2 and 3 hereof and in FIGS. 1 to 4 hereof are simulated process data based on real plant operation data. This Example 2 illustrates how separation of isoparaffin and normal paraffin can be used to debottleneck an alkylation process unit. FIG. 1 hereof shows the base alkylation unit configuration of a refinery producing 13,000 barrels per day (bpd) of alkylation product, an average Butamer™ unit capacity of about 3900 bpd of butane feed, and a total deisobutanizer feed rate of about 70,000 bpd, recycle isobutane purity at about 75%. All numbers in all figures hereof represent bpd. A Butamer™ process is a fixed-bed catalytic isomerization process that typically uses high-activity chloride-promoted catalysts to isomerize normal butane to isobutane. In conventional applications, unconverted normal butane is recycled to extinction through the use of a deisobutanizer column (DIB) or an isostripper column associated with an alkylation unit. FIG. 2 hereof shows that using a rapid cycle pressure swing adsorption to separate isobutane from normal butane, the production of alkylate can be increased by about 1,760 bpd. Rapid cycle pressure swing adsorption is applied to the effluent stream of Butamer™ unit and only the required isobutane is sent to the alkylation unit. Minimizing the amount of normal butane to the alkylation unit frees up the capacity of the alkylation reactor and isobutanizer fractionator to produce about an additional 1,760 bpd of alkylate. The energy requirement of the overall process is also significantly reduced.

The symbols as noted in FIGS. 1-4 are as follows: normal butane (nC₄); iso-butane (iC₄); combined propylene and butylene (C₃C₄⁼); combined propane and butane (C₃C₄); combined C₃, C₄, and C₅ hydrocarbons products (C₃C₄C₅). All values in FIGS. 1-4 are in barrels per day (bpd).

### Example 3

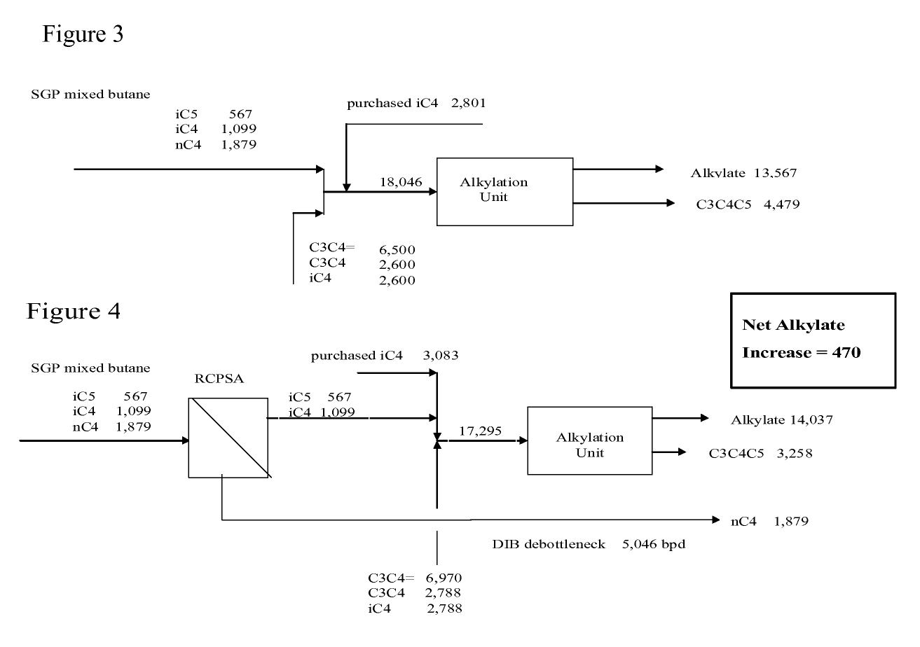

Many refineries do not have a Butamer™ isomerization unit. The feed to the alkylation unit in this case is typically saturated gas purge (SGP) which is a mixture of normal paraffins and isoparaffins. FIGS. 3 and 4 hereof show such a configuration for a petroleum refinery. The rapid cycle pressure swing adsorption unit can be used on the SGP feed to concentrate isobutane that is sent directly to an alkylation unit. Since a limited quantity of normal butane is sent to the alkylation reactor/fractionators, the capacity of alkylation unit is increased by about 470 bpd of alkylate for an SGP feed comprised of about 567 bpd C₅; 1,099 bpd iC₄; and 1,879 bpd nC₄. The capacity of the alkylation unit is about 13,567 bpd of alkylate production without any RCPSA separation of iso/normal components as compared to about 14,037 bpd of alkylate production utilizing the RCPSA separation of the present invention.

The practice of the present invention provides a more cost effective option for separating normal paraffins from isoparaffins and thus provides flexibility to refiners. Blending of isobutane into motor gasoline is limited during the summer months due to Reid Vapor Pressure (RVP) constraints. Many refiners, not having the ability to separate normal butane from isobutene, end up transporting mixed butanes to offsite storage facilities which is then transported back to the refinery during winter months. Refineries incur significant cost as a result of this back and forth transport of mixed butanes. Installing a skid mounted rapid cycle pressure swing adsorption unit at the refinery for separating normal butane and isobutane would allow refiners to reduce this cost because of a much reduced volume of the isobutane portion of the mixed butanes. Normal butane separated by rapid cycle pressure swing adsorption is blended back into the mogas pool all year round.

What is claimed is:

1. A method for the separation of normal paraffins from isoparaffins from a hydrocarbon feedstream that contains both normal paraffins and isoparaffins, which method comprises passing said feedstream to a rapid cycle pressure swing adsorption unit having a total cycle time less than about 1 minute and containing an adsorbent material capable of adsorbing at least an effective amount of normal paraffins from said stream and separately collecting a normal paraffin-rich effluent stream and an isoparaffin-rich effluent stream;
wherein said normal paraffin-rich effluent stream has a higher wt % of normal paraffins than said hydrocarbon feedstream, and said isoparaffin-rich effluent stream has a higher wt % of isoparaffins than said hydrocarbon feedstream and wherein the adsorbent material is selected from the group consisting of crystalline molecular sieves, activated carbons, activated clays, silica gels, activated aluminas and zeolites.

2. The method of claim 1 wherein the cycle time is less that about 30 seconds.

3. The method of claim 1 wherein the adsorbent material is a structured adsorbent material.

4. The method of claim 3 wherein the structure of the structured adsorbent material is selected from monoliths and sheets.

5. The method of claim 1 wherein the adsorbent material is a zeolite with a zeolitic framework selected from the group consisting of CHA, HEU, ERI, FAU, FER, MOR, LTA, and KFI.

6. The method of claim 1 wherein the adsorbent material is a zeolite selected from the group consisting of Chabazite, Linde D, Clinoptilolite, Erionite, Faujasite, Linde X, Linde Y, Ferrierite, ZSM-35, Mordenite, Linde A, and Linde P.

7. The method of claim 6 wherein the adsorbent of the adsorbent material is agglomerated with a binder material.

8. The method of claim 7 wherein the binder material is selected from the group consisting of metal oxides, clays, silicas, aluminas, silica-aluminas, silica-zirconias, silica-thorias, silica-berylias, silica-titanias, silica-aluminas-thorias, silica-alumina-zirconias, and mixtures thereof.

9. The method of claim 8 wherein the binder material is a clay.

10. The method of claim 9 wherein the clay is selected from the group consisting of attapulgite, kaolin, volclay, sepiolite, polygorskite, kaolinite, bentonite, montmorillonite, illite and chlorite.

11. The method of claim 6 wherein the temperature of adsorption is from about 10° to 400° C.

12. The method of claim 11 wherein the temperature of adsorption is from about 90° to about 320° C.

13. The method of claim 11 wherein the pressure during adsorption range from about 50 to about 2000 psia.

14. The method of claim 13 wherein the adsorbent material is a zeolite selected from the group consisting of Chabazite and Linde D, and has a silica to alumina ratio greater than about 1000.

15. The method of claim 13 wherein the feedstream containing both normal paraffins and isoparaffins is an effluent stream from an isomerization process unit and the carbon number of the paraffins ranges from 5 to 9.

16. The method of claim 14 wherein the feedstream containing both normal paraffins and isoparaffins is an effluent stream from an isomerization process unit and the carbon number of the paraffins ranges from 5 to 9.

17. The method of claim 13 wherein the feedstream containing both normal paraffins and isoparaffins is an effluent stream from a Butamer™ process unit containing predominantly C₄ paraffins.

18. The method of claim 14 wherein the feedstream containing both normal paraffins and isoparaffins is an effluent stream from a Butamer™ process unit containing predominantly C₄ paraffins.

19. The method of claim 1 wherein the feedstream containing both normal paraffins and isoparaffins is a saturated gas purge stream.

20. The method of claim 14 wherein the feedstream containing both normal paraffins and isoparaffins is a saturated gas purge stream.
